(19) United States
(12) Patent Application Publication
(10) Pub. No.: US 2010/0316642 A1
(43) Pub. Date: Dec. 16, 2010

(54) COMPLEXES OF GRP94 WITH HUMAN IMMUNOGLOBULIN G

(76) Inventor: Paola Finotti, Padova (IT)

Correspondence Address: MCCORMICK, PAULDING & HUBER LLP, CITY PLACE II, 185 ASYLUM STREET, HARTFORD, CT, 06103, US

(21) Appl. No.: 12/670,335

(22) PCT Filed: Jun. 30, 2008
(86) PCT No.: PCT/EP2008/058375

§ 371 Date: Jul. 2, 2010

(30) Foreign Application Priority Data
Jul. 24, 2007 (IT) PD2007A000252

(51) Int. Cl. A61K 39/395; C07K 19/00; C12N 5/078; C40B 40/10; C40B 30/04; G01N 33/566; A61P 35/00

(52) U.S. Cl. 424/134.1; 530/387.3; 435/375; 506/18; 506/9; 436/501

(57) ABSTRACT

Complexes are described that form in vitro following incubation of “Heat Shock Protein” (HSP) “Glucose-regulated Protein” 94 (Grp94) with human non-immune immunoglobulin G. Results show that complexes of Grp94-IgG are resistant to denaturing agents. Moreover, complexes display an important cytokine-like property that can be exploited to induce positive effects of immuno-modulation in pathologies characterized by either a reduced or exacerbated immune response. In addition, stability of Grp94-IgG complexes make them useful as diagnostic tools to detect antibodies directed against Grp94 in various pathological conditions.

### CROSS REFERENCE TO RELATED APPLICATIONS

This application is entitled to the benefit of and incorporates by reference essential subject matter disclosed in Italian Patent Application No. PD2007A000252 filed on Jul. 24, 2007 and International Patent Application No. PCT/EP2008/058375 filed on Jun. 30, 2008.

### FIELD OF THE INVENTION

The invention regards a new type of complexes that form between Grp94 (“Glucose-regulated protein” 94) and human IgG and the diagnostic and therapeutic uses thereof.

### BACKGROUND OF THE INVENTION

The search for a molecule that could meet the criteria for a useful biological marker of vascular damage in diabetes, being thus an indicator of early and stable alterations that cause open vascular pathologies, led to the discovery in the plasma of type 1 diabetic subjects of some “Heat Shock Proteins” (HSPs) (HSP90, HSP70 and Grp94), among which Grp94 turned out to be the most represented. The study was carried out on pooled plasma obtained from ten type 1 diabetic subjects with diabetes lasting for at least 10 years. It was observed that Grp94 entirely accounted for serine-like proteolytic activity present in diabetic plasma, significantly higher than that of plasma of normal (non diabetic) subjects (Pagetta et al. Diabetologia, 2003; 46:996-1006). Moreover, Grp94 purified from diabetic plasma showed structural and functional properties different from those of native Grp94 purified from cells (Pagetta et al. Diabetologia, 2003, cit. ref.). Thus, not only had plasma Grp94 a higher proteolytic activity than the native counterpart (Menoret et al. J. Biol. Chem., 2001; 276:33313-33318), but it was even structurally different, as revealed by affinity chromatography on the Con-A Sepharose column. However, the most interesting result of the research was the finding that HSPs were present in elevated concentrations in plasma. Indeed, HSPs are exclusively intra-cellular proteins with the main function to chaperon native proteins in order to confer on them the correct folding (Pelham H. R. B. Cell, 1986; 46:959-961; Spiess C. et al. Cell, 1999; 97:339-347; Lee A. S. Trends Biochem. Sci., 2001; 26:504-510). This property is closely related to specific structural determinants located in both N- and C-terminal regions of HSPs that affect binding to peptides and proteins depending on their hydrophobic properties.

It is known that the extra-cellular liberation of HSPs occurs as a consequence of an altered permeability of the cell membrane and/or necrosis of cells, conditions that characterize the inflammatory and immune processes accompanying the development of autoimmune diseases (Finotti P. and Pagetta A. Biochem. Biophys. Res. Commun., 2004; 315:297-305). The un-physiologic extra-cellular location of HSPs confers on them a cytokine-like, inflammatory and immunogenic property characterized by an intense immune response, both humoral and cellular (Asea A. et al. J. Biol. Chem., 2002; 277:15028-15034). This property was indirectly confirmed by the finding that complexes of HSP with IgG were also present in plasma of diabetic subjects (Pagetta A. et al. 2003, cit. ref.; Finotti P. et al. 2004, cit. ref.). Specifically, it was observed that both Grp94 and HSP70 formed high-molecular-mass homo- and hetero-complexes, the latter also containing albumin and α1-antitrypsin (Finotti P. et al. 2004, cit. ref.). The additional observation showing that Grp94 was able to irreversibly link IgG subunits, as demonstrated by experiments of immunoprecipitation (Finotti P. et al. 2004, cit. ref.), corroborated the hypothesis that the irreversible binding engaged by HSPs with circulating proteins led to the formation of fusion proteins, that might be thus responsible for triggering an immune response with the development of auto-antibodies. However, while binding of HSPs to albumin could be considered “aspecific”—given the elevated plasma concentration of albumin and its well-known capacity to reversibly bind a number of various plasma proteins—the supra-molecular aggregates of HSPs (mostly Grp94) with IgG, raised the possibility that these complexes were immune in nature. In fact, not only Grp94 alone but also Grp94 in stable complexes with IgG and α1-antitrypsin might acquire the characteristics of an immunogen, capable of stimulating the production of antibodies (Abs) directed against both Grp94 and the complex itself. This interpretation is corroborated by several reports in the literature showing that in inflammatory and immune diseases, such as essential hypertension and atherosclerosis, the plasma concentration of some HSPs is increased (Xu Q. Thromb. Vasc. Biol. 2002; 22:1547-1559). This finding indirectly demonstrated that, once exposed extra-cellularly, HSPs behave as immunogens (Finotti P. Curr. Diabetes Rev. 2006; 2:295-305). The possibility was also considered that stable complexes of HSPs with both IgG subunits and α1-antitrypsin, instead of being formed in plasma, were liberated as such from the cell, as a consequence of an altered permeability of the membrane and/or cell necrosis. Indeed, it is known that both HSP70 and Grp94 are specifically involved in the assembly of IgG chains into specialized cells (cells of immunity) from which IgG molecules can be secreted only after their subunits are correctly assembled (Melnick J. et al. J. Biol. Chem., 1992; 267:21303-21306). Thus, since the main function of HSPs is to chaperon proteins, it was reasonable to hypothesize that, once liberated into the circulation, HSPs become immunogenic not only for their own structural properties, but mainly for their capacity to form stable, irreversible complexes with other peptides/proteins (Pagetta A. et al. 2003,cit. ref.; Finotti P. et al. 2004, cit. ref.; Finotti P. 2006, cit. ref.).

Experiments conducted on the plasma of other diabetic subjects confirmed and extended previous results, showing that Grp94 actually behaved as a shared immunogen capable of stimulating in any subject the development of primary (idiotypic) anti-Grp94 Abs that also displayed serine-like catalytic activity, being thus considered as catalytic Abs. However, the catalytic activity of anti-Grp94 Abs was exclusively evidenced when complexes with Grp94 underwent disaggregation, whereas integer complexes were catalytically inactive; this was due to the fact that in supra-molecular complexes with both immune and non-immune IgG, catalytic sites were masked (Pagetta A. et al. Mol. Immunol., 2007; 44:2870-2883).

The purification of complexes of Grp94 with idiotypic Abs was obtained by means of Protein G affinity chromatography. However, the isolation and purification of the primary immunogen, i.e. Grp94, from the complex turned out to be technically impossible, due to the fact that in any plasma analyzed (both single and pooled), Grp94 remained tenaciously bound to IgG, regardless of whether IgG were integer or fragmented (Fab and/or Fab₂).

Considering the structural and functional characteristics of both Grp94 and idiotypic Abs in circulating complexes, the hypothesis was raised that these complexes are involved in the development and progression of vascular alterations associated with diabetes.

### SUMMARY OF THE INVENTION

In the attempt to reproduce in vitro the condition found in vivo, i.e., to obtain the formation of complexes similar to those purified from plasma of diabetic subjects, the inventor found that Grp94-IgG complexes could also form in vitro, and that they significantly differ from native immune complexes. In particular, Grp94-IgG complexes obtained in vitro show the following structural and biological properties:
1) Grp94 stably binds human IgG at site(s) other than the antigen-binding site on the IgG molecule (Fab);
2) complexes are resistant to denaturing procedures, such as boiling and reducing agents;
3) complexes have a mass higher than 300 kDa, comprised from 300 to 350 kDa, as demonstrated by experiments of glycerol density gradient centrifugation (gradient from 10% to 40%);
4) complexes show a cytokine-like, immuno-stimulating activity but lack any serine-like catalytic activity.

Therefore, the first object of the invention are complexes of Grp94 with human, non-immune IgG, characterized by:
stable, irreversible binding between Grp94 and IgG at site(s) other than the antigen-binding site on IgG;
resistance to denaturing conditions;
a mass higher than 300 kDa, measured on glycerol gradient (from 10% to 40%).

The non-antigen binding site(s) has/have to be intended as the site(s) occurring in the hinge region of IgG and involving the proximal portion of either Fc, Fab or both.

Yet, in another aspect the object of the invention is also the use of these Grp94-IgG complexes as immuno-modulators (i.e., immuno-stimulants or immuno-adjuvants) for treatment of pathologies in which either the stimulation or inhibition of the immune response is required. The former case relates to conditions in which the immune response turns out to be insufficient (for example, in tumors), whereas the latter pertains to autoimmune diseases in which instead the immune response is exacerbated.

Further objects of the invention are the use of these complexes as diagnostic tools and the methods of modulating the immune response ex vivo.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1. The figure shows the formation of the complex between Grp94 and IgG, demonstrated with different experimental procedures (described in detail in the experimental section): (A) Dot-blot of native Grp94 (2 μg, 10 μl) performed on Immobilon membranes previously soaked in methanol (for 1 min), rinsed with distilled water (for 1 min) and dried out for 3 min. (B) SDS-PAGE and Western blotting with anti-Grp94 monoclonal and anti-human IgG polyclonal (from sheep) Abs on native Grp94 after the final step of purification on the Con-A Sepharose column, in both the absence and presence of human non-immune IgG (at the molar ratio of 1:1 with Grp94), after 1-h incubation at 37° C. (C) Fractional centrifugation on glycerol density gradient (from 10% to 40%) of Grp94 (100 μg) after incubation for 1 h at 37° C., in both absence and presence of human non-immune IgG (at the molar ratio of 1:1).

FIG. 2. The figure shows the effects of Grp94, both alone and in complexes with IgG, on both cell growth and angiogenic differentiation of human umbilical vein endothelial cells (HUVECs). (A) Histograms represent the mean (±SEM, of at least three different experiments) of the cell number counted after incubation of cells in the absence (control) and presence of Grp94 (1 and 10 ng/ml), both alone and in complexes with IgG. (B) Histograms represent the mean (±SEM, of at least three different experiments) of the cell number counted after incubation as in (A) with the addition of the MEK (Mitogen-activated protein kinase kinase) inhibitor U0126 (10 μM, final concentration). (C) Pictures taken at the optical microscopy showing angiogenic transformation of HUVECs after the treatment with Grp94 (10 ng/ml), both alone and in complexes with IgG, with and without U0126 inhibitor.

FIG. 3. The figure shows the expression of both HSP90 and HSP70 after the treatment with Grp94, both alone and in complexes with IgG in HUVECs. (A) SDS-PAGE (10% acrylamide gel) and Western blotting with anti-HSP90, anti-HSP70 and anti-human IgG Abs of whole cell lysates after the treatment with Grp94 (10 ng/ml), both alone and in complexes with IgG, in both the absence and presence of the inhibitor U0126 (10 μM, final concentration). Arrows on left mark the bands also shared by nearby lanes on right. Western blotting is representative of three other analyses made on different occasions on the same samples. (B) SDS-PAGE (10% acrylamide gel) and Western blotting with anti-HSP90, anti-HSP70 and anti-human IgG Abs (as above, for whole cell lysates) of conditioned media harvested from the same cell cultures used for the experiments described in (A).

FIG. 4. The figure shows structural changes of HUVECs mediated by HSP90 and HSP70 after treating cells with Grp94, both alone and in complexes with IgG. (A) The green fluorescence evidenced at the confocal microscopy with anti-HSP90 Abs is shown in both control and treated cells (panels a, b, c on left). (B) Green fluorescence due to HSP70 after treatment with anti-HSP70 Abs (as for HSP90).

FIG. 5. The figure shows the growth stimulation of human peripheral blood monocyte cells (PBMCs) in the presence of Grp94 alone and in complexes with IgG.

FIG. 6. The figure shows the stimulation of IgG production from PBMCs in cultures with Grp94, both alone and in complexes with IgG.

### DETAILED DESCRIPTION OF THE INVENTION

The experiments aimed at obtaining the formation of Grp94-IgG complexes in vitro originated from the necessity to reproduce, in a simplified experimental model, the complex condition of native, immune complexes found in ex vivo experiments of purification from diabetic plasma. If complexes similar to those found in vivo between Grp94 and IgG could also form in vitro, then, it was possible to study in detail the mechanism(s) by which Grp94 and IgG, both singularly and combined together, displayed their effects on HUVECs, taken as a model of endothelial vascular cells. It is clear, however, that marked differences exist between the in vitro and in vivo conditions that affect the formation of Grp94 and IgG complexes.

Differences are:
1) Grp94 used in experiments in vitro is obtained from the microsomal fraction of rat hepatocytes. Although antigenic sites in rat Grp94 are similar to those of human native Grp94 (anti-Grp94 Abs recognize both human plasma and rat Grp94), it is expected that rat Grp94 partly differs from Grp94 found in plasma of diabetic subjects (Pagetta A. et al. 2003, cit. ref.);
2) human IgG employed in experiments in vitro are non-immune in nature (being obtained from a pool of plasma from normal subjects).

In addition, results of experiments showed that:
3) the Grp94-IgG complex obtained in vitro forms after a relatively short time of incubation of the two reactants; this suggests that sites on both Grp94 and IgG react rapidly with each other in a chemical reaction that leads to the formation of a stable bond;
4) initially, binding may be still reversible, whereas reversibility is rarely (or even never) encountered in vivo, since Grp94 can be hardly detached from IgG in native complexes;
5) the characteristics of binding in experiments in vitro are dependent on different variables, such as, among others, the concentration of reactants, time of incubation and temperature, significantly different from variables present in vivo.

The Grp94-IgG complexes, object of the invention, are obtained by co-incubating native or recombinant mammalian Grp94 with human, non-immune IgG at molar ratios comprised between 0.5 and 1.0 (Grp94 to IgG, M:M), preferably at the molar ratio of 1:1, at temperatures between 30 and 40° C., preferably at 37° C., for at least 1 h in aqueous solvent, optionally buffered (pH between 6.5 and 7.4).

As far as the aim of the invention is concerned, Grp94 is the mammalian protein P14625 (identification code of the protein in SwissProt & Tremble data bank). Since Grp94 is a phylogenetically highly conserved intra-cellular protein, and the human variant shows significant homology of sequence (>80%) with both rat and mouse Grp94, it follows that rat and/or mouse Grp94 can be used advantageously in place of human Grp94.

IgG are always human and non-immune in nature, i.e., obtained from the plasma of normal subjects and purified with well-known standard procedures.

Complexes form after the co-incubation of Grp94 with non-immune IgG and show the characteristics reported above, that is: i) a stable binding that does not involve the antigen-binding site on the IgG molecule, occurring in the hinge region of IgG and involving the proximal portion of either Fc, Fab or both; ii) resistance to denaturing conditions, such as boiling and reducing treatments; iii) a mass higher than 300 kDa, more specifically comprised between 300 and 350 kDa, as determined in the experiments of centrifugation on glycerol density gradient from 10% to 40% using integer IgG molecule.

Besides these structural characteristics, complexes formed in vitro also show peculiar unexpected biological properties. Complexes are able:
1) to stimulate the cell growth and to induce the differentiation of HUVECs towards the capillary formation;
2) to enhance the expression in HUVECs of both HSP70 and HSP90 that mediate structural changes of the cytoskeleton with the formation of numerous podosomes;
3) to induce the expression and secretion from HUVECs of IgG;
4) to stimulate the proliferation of human PBMCs associated with a significant increase in the IgG production.

In experiments of dot-blot it was possible to establish that native Grp94 was able to bind steadily IgG only if not denatured, whereas after denaturation (for example, after boiling) Grp94 lost this capacity. It was thus apparent that binding takes place at sites on IgG different from those involved in antigen binding (Fab), and that complexes are non-immune (being formed with non-immune IgG). Complexes have a mass of 300-350 kDa (as determined in experiments of centrifugation on glycerol density gradient using integer IgG molecule) and are stable even in denaturing and reducing conditions, such as boiling and treatment with β-mercaptoethanol. In addition, these complexes display important biological effects on HUVECs, including the stimulation of cell growth, induction of angiogenic differentiation and the increased expression of HSP90 and HSP70. Complexes also stimulate the proliferation of human PBMCs already at a very low concentration (1 ng/ml). The pattern of stimulation of both HUVECs and PBMCs overlapped that displayed by various growth factors, being thus characterized by the appearance of the stimulatory effect in a narrow range of concentrations, with maximal effect peaking at a very low concentration (in the nanomolar range). The stimulation of PBMC proliferation by Grp94-IgG complex was also accompanied by a significant increase in the production of IgG, particularly evident at the concentration of 10 ng/ml.

Since the effects observed with complexes formed in vitro overlapped those caused by cytokines, complexes are expected to behave like cytokines in affecting the immune response; complexes are thus exploitable as therapeutic agents in vivo, in conditions in which the immune response needs modulation.

Results of experiments described in detail hereinafter, suggest an important therapeutic application for complexes of Grp94 with human non-immune IgG of the invention as immuno-modulators in pathologies in which either a stimulation or inhibition of the immune response is required, the former being referred to conditions characterized by a deficit of the immune response (as tumors, inflammatory diseases, immune deficiency of different etiologies), the latter comprising autoimmune diseases in which there is an exaggerated immune response that must be repressed.

As far as the former condition is concerned, complexes of Grp94 with IgG can be employed as a new therapeutic vaccine in the treatment of tumors, similar to cytokines capable of inducing a strong humoral and cellular immune reaction. It is known that the vast majority of tumors lack immunogenicity and are thus appropriate targets for an active immunization by vaccination (Raez L. E. et al. Clin. Med. Res., 2005; 3:221-228). The Grp94-IgG complexes, according to the invention, may be used as a vaccine in which Grp94 is rather the antigen and IgG the adjuvant, capable to enhance the immune response (i.e., the production of anti-Grp94 Abs specifically evoked by Grp94), also favoring a strong differentiation effect. The rationale for the use of the Grp94-IgG complexes as effective tumor vaccine is based on the common knowledge that the expression of Grp94 is increased in cancer cells, representing a common cell surface antigen in several tumors (Takagi S. et al. Hum. Pathol., 2004; 35:881-886; Zhu X. D. et al. World J. Gastroeneterol., 2004; 10:1141-1145; Akutsu Y. et al. Int. J. Oncol., 2007; 31:509-515). However, although the extra-cellular expression of Grp94 is increased in almost any cancer cells, in the majority of cases this expression is insufficient to evoke an efficient immune response that leads to tumor rejection. Vaccines so far available with Grp94 are all made with Grp94 in complexes with various peptide(s), mostly those originating from tumor cells (Chandawarkar R. Y. et al. Int. Immunol., 2004; 16:615-624; Liu B. et al. Vaccine, 2008; 26:1387-1396). In these known vaccines, Grp94 is included as adjuvant, whereas peptide(s) represent true antigen(s) that are responsible for specific immunization (raised against tumor) (Dai J. et al. Cancer Immunity, 2003; 1:1-11). However, many obstacles hamper the successful development of vaccine therapy based on the principle of tumor-derived peptides as true antigen(s). First, peptides differ in both the nature and quantity, depending on the differentiation stage of cells forming primary and/or secondary (metastatic) tumor(s). Immunogenicity of peptides may thus change significantly during different phases of the tumor development (Jäger E. et al. Curr. Opin. Immunol., 2002; 14:178-182) rendering vaccination ineffective. Second, a tissue- and/or organ-specific tumor is expected to display its own specific set of antigenic peptides, this fact implying that individual vaccine therapies are necessary for any specific type of tumors. Third, for obtaining antigenic peptides, it is obligatory that tumor can be excised and a series of time-consuming and expensive procedures of purification made on a sufficient number of tumor cells. All these conditions heavily restrict the margins to an effective and on a-large-scale therapeutic application of tumor vaccines.

The Grp94-IgG complexes that are object of the invention could represent a significant improvement in the effort to develop a reliable and efficient anti-tumor therapy: it combines the specificity of the immune response, directed against an antigen (Grp94) that is shared by any cancer cells, regardless of their nature, stage of differentiation and location, with the capacity of the IgG molecule (adjuvant) to confer on the complex an enhanced capacity to stimulate the immune system to produce specific Abs.

Furthermore, Grp94-IgG complexes of the invention can also be usefully employed as immuno-modulators in the “negative” vaccine therapy for autoimmune diseases in which an exaggerated immune response is directed against self antigen(s). The exploitation of Grp94-IgG complexes as “negative” vaccine, i.e., with the capacity to inhibit the immune activation, originates from the premise that, similarly to what happens in cancer cells, cell surface presentation of Grp94 also takes place in the autoimmune process. However, both the entity of the expression on the cell membrane and the extra-cellular release of Grp94 crucially dictate the nature of the immune response, whether tolerant or auto-directed. Thus, a rapid increase in the concentration of cell membrane-bound or soluble Grp94 causes a shift in the response that from tolerant becomes auto-directed, against cells presenting the antigen. The knowledge of the very first antigen in the development of autoimmune process is the prerequisite for obtaining an effective vaccine. Thus, the premise for using the Grp94-IgG complexes, object of the invention, as “negative” vaccine in various autoimmune diseases is that Grp94 is the most important part of (if not even the only) antigen presented on the cell membrane in the pathological conditions mentioned above. One possible mechanism by which the autoimmune process could be arrested predicts that the Grp94-IgG complex is administered at a stage of disease in which there is an intense activation of the immune response specifically sustained by the sub-set of T helper-1 (Th-1) lymphocytes. In this condition, it is known that an elevated dose of vaccine can suppress the autoimmune aggression by causing a shift in the population of lymphocytes that from Th-1, inflammatory, turn into Th-2, tolerant (Ghoreschi K. Trends Mol. Med., 2003; 9:331; Steinman R. M. J. Clin. Inv., 2002; 12: 1519; Chandawarkar R. Y. et al. 2004, ref. cit.).

As far as the therapeutic intervention is concerned, complexes of Grp94 with human non-immune IgG of the invention, can be thus profitably employed in both the prevention and treatment of pathologies characterized by either a deficit, or an excessive stimulation of the immune response. In the Grp94-IgG complex, the Grp94 can be either a native mammalian protein or a recombinant protein thereof, both integer or fragment or a mimetic of it, whereas IgG or fragments thereof, either Fab or Fc portions, are human and non-immune, obtained from plasma of healthy donors.

Therefore, for the purpose of the invention the Grp94-IgG complexes can be formed with integer IgG or fragments thereof, whereas the Grp94 in the complex can be the integer molecule or fragments or a mimetic thereof (such as for example HSP 90).

The Grp94-IgG complexes of the invention can be employed as immuno-modulators prepared in pharmaceutical compositions with the addition of appropriate excipients, carriers and/or diluents. To this aim, the pharmaceutical formulations containing the Grp94-IgG complexes are those suitable for systemic and/or local administration also in suitable, controlled-release delivery systems. The pharmaceutical formulations also include those developed with either standard procedures or innovative technologies, using recently designed biomaterials and other materials, additives, diluents, emulsifiers, aqueous and oily or even polymeric vehicles, all suitable for pharmaceutical employment.

The suitable pharmaceutical formulations to be used in the parenteral administration are those already known, such as ready-to-use vials containing the pharmacologically active substance in solution or suspension, or as lyophilized powder to be diluted with aqueous (either buffered or with appropriate suspension particles) or oily solvents added at the moment of administration.

The amount of the active substance present in each formulation, as well as the therapeutic dosage will vary depending on the pathology and its severity, on the risk of developing the pathology itself and on general wealth conditions of the patient, including his/her body weight. The amount of the active substance may be comprised between 0.02 mg/kg and 5 mg/kg of body weight, administered in a single or multiple daily dosages, at different time intervals in repeated cycles of therapy.

The Grp94-IgG complexes of the invention can be further used in ex vivo experiments aimed at modulating the immune response. Thus, isolated cells of the immune system, such as lymphocytes, antigen-presenting cells, dendritic cells, can be isolated from the patient blood and co-incubated with the complexes of the invention for at least 1 h. Preferably, cells of the immune system are a sub-population of cells selected on the basis of a specific antigen and/or membrane marker before the incubation with the Grp94-IgG complexes of the invention. The preferred concentration of cells is 2 million/ml, while the complexes can be used at concentrations comprised between 10 ng/ml and 10 μg/ml.

Furthermore, considering that: a) the concentration of anti-Grp94, anti-HSP70 and anti-HSP90 primary (idiotypic) Abs is increased in the plasma of diabetic subjects, as well as in other inflammatory pathological conditions, both acute and chronic, and that these Abs are present independently of the duration of disease and in concentrations that varies from subject to subject; b) in the plasma of type 1 diabetic subjects also circulate Abs directed against the immune (native) Grp94-IgG complex (anti-idiotypic or secondary Abs), it follows that both primary and secondary Abs are markers of inflammation closely linked to an altered immune response. The detection of these Abs, both idiotypic, against Grp94, and anti-idiotypic, against the immune Grp94-IgG complex, can be exploited as diagnostic and prognostic marker of inflammation correlated with various inflammatory/metabolic diseases, such as diabetes, essential hypertension, atherosclerosis, alterations of lipid metabolism of different etiologies, in autoimmune diseases, such as systemic lupus erythematosus, rheumatoid arthritis and allergic diseases, and in infectious diseases (by Mycobacterium tuberculosis, Chlamydia and virus) in which Grp94 has been recognized to have an etiopathogenetic role in inflammatory reactions in the vascular bed. In particular, the detection of the above mentioned Abs at significant concentrations in apparently still healthy people might predict the development of disease (having thus a diagnostic meaning), whereas changes in the concentration of Abs in already overt disease might be related to either remission or exacerbation of disease-associated inflammatory complications (thus having a prognostic meaning).

Thus, Grp94-IgG complexes that are object of the invention could be profitably employed in diagnostic kits for the measurement of anti-idiotypic Abs (recognizing the Grp94-IgG immune complex), whereas idiotypic Abs could be detected by using denatured Grp94 as antigen.

The detection of Abs could therefore regard:
a) anti-idiotypic Abs developed against native Grp94-IgG complex. Anti-idiotypic Abs may recognize the Grp94 or IgG portion of the complex, or both. These Abs could be revealed by binding the Grp94-IgG complexes, or a fragment, analog, derivative or mimetic thereof, according to the invention, to appropriate array substrates, selected among micro-wells, membranes, resins or gels and preferably selected in the group of glass or silicon chips, plastic micro-wells, nitrocellulose membranes or resins attached to glass slides, three-dimensional gels made of cross-linked polymer containing reactive groups;
b) idiotypic anti-Grp94 Abs that could be detected using native (or recombinant) Grp94 after denaturation.

As far as the point a) is concerned, the quantitative determination of plasma/serum Abs can be performed with the method here below described (direct labeling, single-Ab assay):

a plasma/serum sample (or the IgG fraction rapidly purified from plasma by means of ion-exchange spin column) obtained from blood of subject(s) who need to be checked in longitudinal and/or cross-sectional studies;

the sample of plasma or plasma-purified IgG fraction is directly labeled with appropriate probes that tag proteins and are capable of generating a measurable signal (colorimetric, fluorescent, chemiluminescent, radioactive);

directly labeled proteins of plasma sample or plasma-purified IgG are placed on the suitable array substrates selected among micro-wells, membranes, resins or gels (preferably selected in the group consisting of micro-wells, membranes, resins or gels, as specified above) on which the Grp94-IgG complexes, or a fragment, analog, derivative or mimetic thereof, according to the invention, has previously been allowed to attach. In particular, attachment to the substrate of the Grp94-IgG complex of the invention, should involve the C-terminal portion of the IgG molecule or a portion on it other than the antigen-binding site or sites specifically involved in Grp94 binding, either directly or indirectly through a spacer, so that a significant part of both Grp94 and IgG forming the complex could be free to interact with Abs directed against the complex;

labeled proteins that do not interact with the attached complex are removed by washings, whereas Abs that specifically recognize the complex can be easily detected by measuring signal generated by the specific probe(s) attached to IgG.

As far as the point b) is regarded, the quantitative determination of plasma/serum Abs can be performed with the method here below described (dual Ab sandwich assay):

a plasma/serum sample (or the IgG fraction rapidly purified from plasma by means of ion-exchange spin column) obtained from blood of subject(s) who needs to be checked in longitudinal and/or cross-sectional studies;

a sample of plasma or plasma-purified IgG fraction is placed on the suitable array substrate (micro-wells, membranes, resins or gels, as specified above) on which denatured Grp94 (either native or recombinant) has been allowed to attach;

after washing away irrelevant proteins, idiotypic (primary, anti-Grp94) Abs are detected by means of tagged detector Abs (for example, anti-human IgG, biotin-labeled Abs) followed by incubation with labeled read-out Abs (for example, anti-biotin Abs), or by means of other standard procedures or other strategies for enhancing immune detection.

Diagnostic kits for the determination of both idiotypic and anti-idiotypic (against the native, immune complex of Grp94-IgG) and idiotypic (anti-Grp94) Abs can comprise: an array format made of a specific substrate (planar glass slides, membranes, plastic micro-wells for enzyme-linked assay, gel-based arrays) on which complexes of the invention, Grp94-IgG, and/or denatured Grp94 (either recombinant or native) have been attached; samples of test proteins for the calibration curve, a reagent solution for labeling sample proteins, a sample of human plasma/serum or plasma-purified human IgG as control together with a brochure with instructions for use.

In particular, the diagnostic kit can comprise at least in one or more containers: a) complexes of Grp94-IgG, or fragment, analog, derivative or mimetic thereof, optionally labeled, immobilized on a suitable array substrate (preferably selected in the group of micro-wells, membranes, resins or gels); b) denatured Grp94 (either native or recombinant), optionally labeled, either in solution or immobilized on a suitable array substrate (preferably selected in the group of micro-wells, membranes, resins or gels); c) test mammalian anti-Grp94 antibodies (preferably from rat, mouse or rabbit) optionally labeled; d) control plasma/serum or plasma-purified IgG fraction; e) reagents for labeling plasma proteins; f) instructions for use.

Here below is described in detail the preparation of complexes of the invention and their structural and functional characterization.

Experimental Part

A-Preparation of Complexes of Grp94 with Human IgG

The first step for obtaining the formation of complex included the purification of Grp94 from the microsomal fraction of rat hepatocytes. Fractions were then submitted to a DEAE-Sepharose column followed by a Heparin-Sepharose column. The Grp94-containing peak, eluted from the Heparin-Sepharose column at 0.5 M NaCl, was chromatographed on FPLC-Superdex 200 (10 mm×300 mm) previously equilibrated with buffer A (20 mM Tris-HCl, pH 7.5, 5 mM magnesium acetate, 1 mM EGTA, 10 mM β-mercaptoethanol, 0.1% Triton X-100, 10% glycerol, 0.05 mM PMSF, 0.02% NaN₃) containing 500 mM NaCl. Fractions of 0.2 ml eluted at a flow rate of 0.4 ml/min. The Grp94-containing fractions were collected and passed through a Con A-Sepharose column (5 ml) previously equilibrated with buffer B (20 mM Tris-HCl, pH 7.5, 1 mM MgCl₂, 1 M CaCl₂, 10 mM β-mercaptoethanol, 10% glycerol, 0.05 mM PMSF). Grp94 was subsequently eluted with buffer B containing 0.6 M α-D-methylmannoside, and its purity tested by immunoblotting with specific Abs.

The Grp94 preparation was also submitted to the QCL-1000 chromogenic LAL end-point assay to exclude any endotoxin contamination.

The purified Grp94 preparation was dialyzed on Spectrapor membrane tubing of 3,500 MWCO overnight at 4 C against Tris buffer (20 mM Tris-HCl, pH 7.5) and then submitted to ultra-filtration on Amicon Centriplus YM-3 of 3,000 MWCO. The protein concentration was measured with the method of Bradford (Bradford M. M. Anal. Biochem., 1976; 72:248-254). Samples of purified Grp94 were stored at −20 C in 50-μl aliquots ready to use.

The purity of human non-immune IgG was assessed by Western blotting with sheep anti-human whole IgG polyclonal Abs and goat anti-Fab polyclonal and mouse γ chain-specific monoclonal Abs. In experiments aimed at evaluating complex formation with IgG, Grp94 was co-incubated with human IgG at 1:1 molar ratio at 37 C for 1 h, in aqueous solvent (final volume of incubation: 100 μl, pH 6.5).

B-Structural Characterization of Grp94-IgG Complexes: Dot-Blot Analysis, SDS-PAGE and Western Blotting, Sedimentation Velocity Analysis

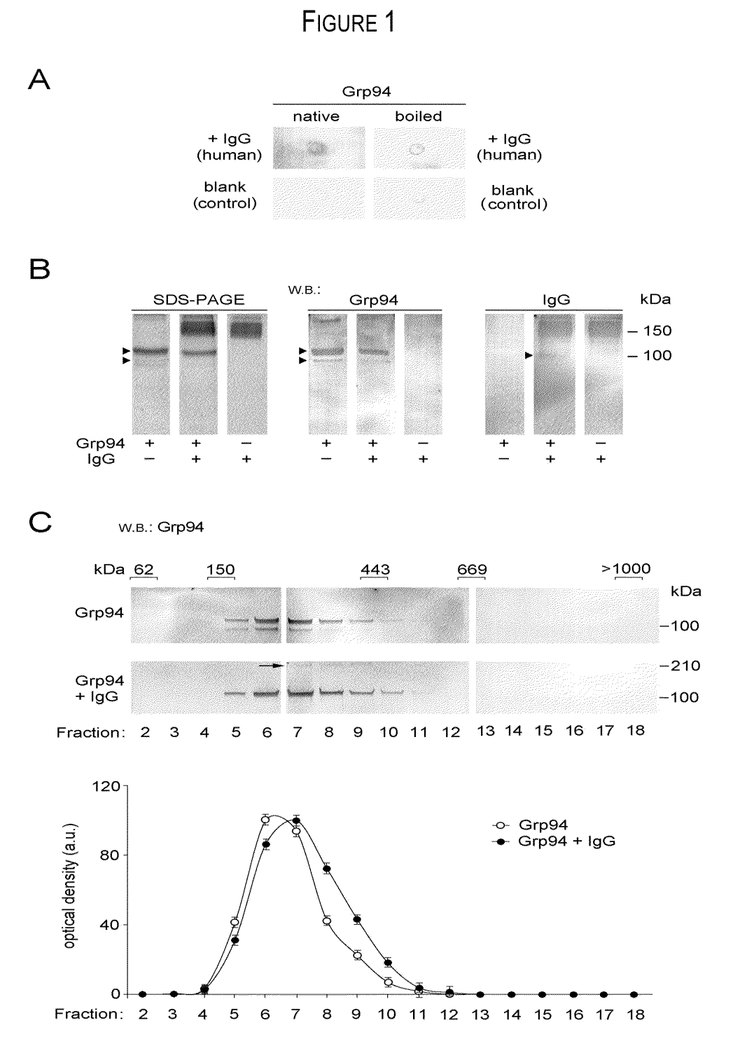

1. Dot-blot analysis: hereafter are described methodological procedures used for the characterization of complexes of Grp94 with human IgG obtained in vitro, as specified above, together with results shown in FIG. 1(A).

In dot-blot experiments, strips of PVDF membrane were rinsed first in methanol (1 min) and then in de-ionized water (1 min) and let partially dry (3 min). Dots were made with 10 μl of Grp94 (2 μg). Grp94 solution (in bi-distilled water) was used as such (without any treatment) or after boiling for 2 min. Dots with each of the Grp94 solutions (both untreated and boiled) were made in duplicate on membrane strips that were incubated in both absence (control, in TBS only) and presence of human IgG (4 μg/ml) for 8 h at room temperature. Membrane strips were then incubated overnight with a blocking solution of BSA (1%) and Tween (0.2%), followed by washings with TBS (for 1 h) and a further incubation with primary sheep anti-human IgG Abs (2.5 μg/ml). After repeated washings with TBS (for 1 h), membrane strips were then immersed in the solution of secondary Abs (donkey anti-sheep IgG Abs) for 1 h and probed with the detection solution. Control strips with dots of Grp94 incubated with TBS alone were probed with both primary and secondary Abs, and with secondary Abs alone. In FIG. 1(A) dots of Grp94 that reacted with anti-human IgG Abs are shown, indicating the formation of stable binding between untreated Grp94 and IgG, whereas dots of control Grp94 (probed with both primary only and primary plus secondary Abs) show only a weak, if not even absent, positivity. Its is also evident that Grp94 after boiling did not bind IgG any longer, thus demonstrating that the formation of complex in vitro only occurred when Grp94 maintained its native conformation.

2. SDS-PAGE and Western blotting: hereafter is described in detail the method used for the characterization of complexes of Grp94 with human IgG obtained in vitro, as specified above, together with results shown in FIG. 1(B). SDS-PAGE was run at 10% acrylamide gel and 2.5 μg of native Grp94 in absence of reducing treatment and boiling was loaded in each lane. Grp94 was detected in two bands at 105 and 92 kDa representing two species with a different degree of glycosylation. Indeed, by submitting Grp94 to de-glycosylation with N-glycosidase F (that removes saccharide residues linked to asparagines in the protein), the band at 105 kDa acquired a higher mobility, focusing at 104 kDa, a mass consistent with the removal of about six mannose residues (data not shown). After incubation, the mobility of Grp94 in the gel remained unaltered with respect to that of the fresh solution, whereas after incubation with IgG, the band of Grp94 at 105, and mostly that at 92 kDa, showed a marked reduction in intensity. This effect was even more evident in Western blotting with anti-Grp94 Abs, thus suggesting that the Grp94 monomer (mostly that with a lower degree of glycosylation) was involved in binding IgG. By probing the same samples of Grp94, previously tested with anti-Grp94 Abs, also with anti-IgG Abs, it was noted that in the sample of Grp94 incubated with IgG a band at about 100 kDa was apparent, not present in the sample of IgG alone (arrow).

3. Glycerol density gradient centrifugation: hereafter is described in detail the method used for the characterization of complexes of Grp94 with human IgG obtained in vitro, as specified above, together with results shown in FIG. 1(C).

In experiments aimed at evaluating the complex formation, Grp94 (200 μg/ml) was co-incubated with human IgG (at the 1:1 molar ratio) at 37 C for 1 h. Control solutions of Grp94 and IgG, at the same concentration as those used in the co-incubation experiment, were also incubated individually. A 240 μl aliquot of each of the incubated sample solutions (100 μg proteins) were subjected to glycerol density gradient centrifugation with 10-40% glycerol gradient in 25 mM Hepes buffer (pH 7.4), containing 1 mM EDTA and 1 mM dithiothreitol. After centrifugation at 100,000×g for 18 h in a Beckman SW60Ti rotor at 4° C., the gradient was separated into 18 fractions of 200 μl each, submitted to SDS-PAGE (10% polyacrylamide gel) followed by Western blotting with anti-Grp94 and anti-IgG Abs. Glutamate dehydrogenase (62 kDa), alcohol dehydrogenase (150 kDa), apoferritin (443 kDa) and thyroglobulin (669 kDa) were used as standards for estimating the molecular mass of the complex.

The method of glycerol density gradient centrifugation is currently used as a valid alternative to gel-filtration for calculating the mass of various protein complexes (Tanahashi N. et al. J. Biol. Chem., 200; 275:14336-14345; Tibaldi E. et al. Cell. Mol. Life Sci. 2006; 63:378-389; Hirano Y. et al. Nature, 2005; 437:1381-1385).

The immunoblot analysis of fractions collected after centrifugation and probed with anti-Grp94 Abs showed that, after incubation, Grp94 alone formed supra molecular complexes, mostly peaking in fraction 6, with an approximate mass of 200-300 kDa, as established on the mass of calibration proteins. This result was in accord with previously reported observations showing that Grp94 monomer is prone to form homo-aggregates (dimers and even bigger complexes). As shown in FIG. 1(C), Grp94 monomer is easily removed from complexes so that it is visible in two main bands, at about 105 and 92 kDa, after reducing treatment with β-mercaptoethanol, likely corresponding to the native species of Grp94 present in hepatocytes at the moment of purification (see above), probably characterized by a different degree of glycosylation. At variance with what observed with Grp94 alone, in the presence of IgG, complexes of Grp94 peaked in a fraction at a higher glycerol density (fraction 7), a finding demonstrating that complexes have a mass higher than 300 kDa. Moreover, after co-incubation with IgG, Western blotting with anti-Grp94 Abs showed the positivity for Grp94 at a molecular weight consistent with the formation of a tenacious complex with IgG (at about 210 kDa), resistant to reducing treatment with β-mercaptoethanol and boiling. It was also apparent that the band of Grp94 at about 92 kDa disappeared after co-incubation with IgG, suggesting possible involvement of this species of Grp94 in binding to IgG. This possibility was further validated by SDS-PAGE (10% polyacrylamide gel, without treatment of samples with β-mercaptoethanol) of Grp94 after incubation in both absence and presence of IgG, before the solutions were submitted to glycerol density gradient centrifugation. FIG. 1(B) shows that, whereas bands of Grp94 at masses lower than 100 kDa are still present (although with a lower intensity) after incubation in absence of IgG, the co-incubation with IgG led to disappearance of the 92-kDa band, the only visible band being that at 105 kDa. Numbers below Western blotting in FIG. 1(C) refer to the fractions collected, while numbers above indicate the mass of standard proteins used for calculating the mass of complexes. On right in the panel with Western blotting are masses (in kDa) of bands of Grp94 immunoreacting with anti-Grp94 Abs. It was apparent that in any fraction of the control solution of Grp94 the positivity was always present in two bands (similarly to what observed with the same solution of Grp94 analyzed before the fractionation on glycerol density centrifugation), whereas after co-incubation with IgG the band of Grp94 at 92 kDa was always absent. Furthermore, the peak of optical density of bands of Grp94 measured in any fraction was shifted towards a fraction at higher glycerol density for Grp94 after co-incubation with IgG (fraction 7) compared with what observed with Grp94 alone (fraction 6), a result that indicated the formation of a complex between Grp94 and IgG. This was further confirmed by the presence of a band at masses higher than 200 kDa in fractions 7-11 that did not change mobility even after reducing and boiling treatments of samples in SDS-PAGE. This band, that testified the presence of an irreversible complex, was instead absent in the same fractions after fractionation on glycerol density centrifugation of Grp94 alone.

C-Characterization of the Biological Activity of Complexes of Grp94 with IgG: Effects on HUVECs and Human Lymphocytes.

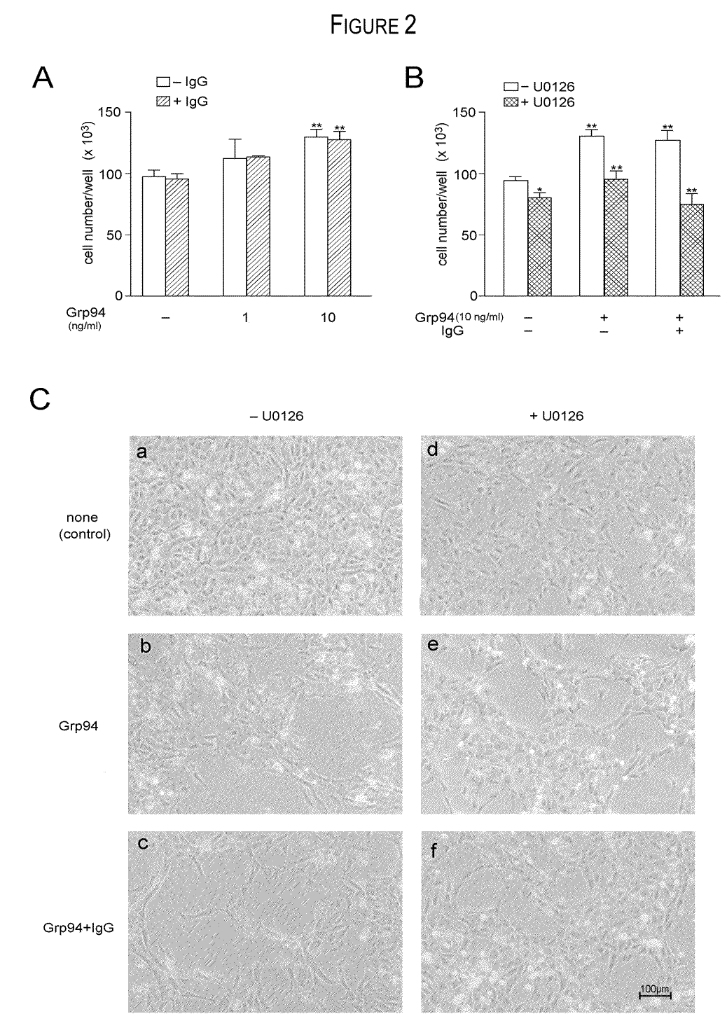

1. Cell growth stimulation and differentiation of HUVECs: hereafter is described in detail the method used for the biological characterization of complexes of Grp94 with human IgG obtained in vitro, as specified above, together with results shown in FIG. 2.

Cells were grown in endothelial basal medium supplemented with fetal bovine serum (FBS) at 10%, and after a starving period of 8-10 h in absence of FBS, fresh serum-free medium was added (2 ml) together with Grp94 (at the final concentration of 1 and 10 ng/ml) both alone and with IgG (at the molar ratio of 1:1). After an incubation period of 20 h, the medium was collected and cells detached from duplicate wells and counted in a hemocytometer. Cell viability was evaluated with the trypan blue dye exclusion method; trypan blue only enters cells with an altered, but not intact membrane permeability.

In FIG. 2 are shown results described hereafter. Histograms in FIG. 2(A) represent the cell growth stimulation (number of cells/well) induced by Grp94 (both alone and with IgG) at the two concentrations compared with the growth of control cells in absence of IgG (97.38±5.9×10³), not different from that obtained in the presence of IgG alone (96.33±5.43×10³). Histograms in FIG. 2(B) represent the number of cells grown with Grp94 (10 ng/ml), both alone and in the presence of IgG, with the addition of the inhibitor U0126 (10 μM, final concentration). Control cells were treated with the diluent alone in which the inhibitor has been dissolved (DMSO 0.1%). The inhibitor was added to cell cultures 30 min before the addition of Grp94, both alone and with IgG. The number of control cells in both absence and presence of the inhibitor was 98.01±5.4×10³ and 80.38±4.02×10³, respectively. Asterisks on bars mark statistically significant (*, p<0.05) and highly significant (**, p<0.001) differences with respect to control values (empty bars).

Microscopic evaluation of morphogenic transformation of HUVECs after Grp94 treatment (FIG. 2, (C)), with and without IgG, in both absence and presence of the inhibitor U0126, shows that angiogenic modifications induced by Grp94, both alone (panel b) and with IgG (panel c), did not change significantly after the addition of the inhibitor (panels e, f), whereas these pictures significantly differed from the cobblestone morphology of control HUVECs (panel a). On its own the inhibitor caused changes in the normal morphology of HUVECs consistent with a pre-angiogenic transformation (panel d).

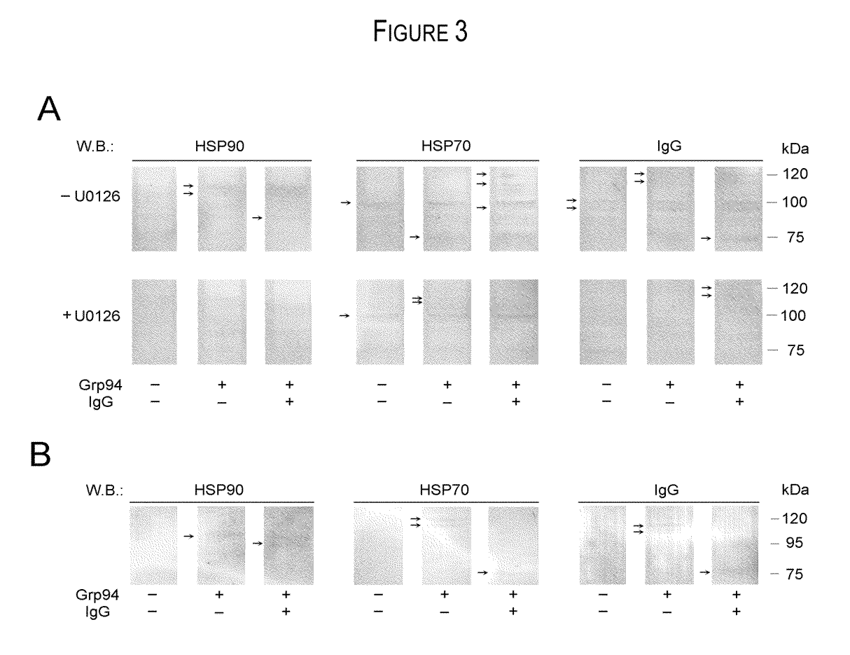

2. Effects on the expression of HSP70 and HSP90 in HUVECs: hereafter are described in detail the method and results shown in FIG. 3(A) referred to experiments performed after 20-h incubation of cells in the presence of Grp94 (10 ng/ml), both alone and with IgG, with and without the inhibitor U0126 (10 μM, final concentration). Cells were detached from wells and treated with lysis buffer (50 mM Tris-HCl, pH 8.9, 5 mM EDTA, 380 mM glycine, 2% SDS, 7 mM β-mercaptoethanol). Whole cell lysates were analyzed in SDS-PAGE (10% polyacrylamide gel) followed by Western blotting with anti-HSP90, anti-HSP70 and anti-human IgG Abs. The same quantity of proteins (determined by means of densitometric analysis made on common bands in any lysates) was loaded in each well in SDS-PAGE. Arrows on left mark bands in the lanes that are also shared by nearby lanes on right. The positivity for HSP90 is only present in treated (with Grp94, both alone and with IgG) but not control lysates, whereas a constitutive form of HSP70 is present in any lysates, although only in treated cells there are additional bands referred to inducible species of HSP70. It is worth noting that band positive for IgG appear at the same masses at which also focuses HSP70. In the presence of the inhibitor, the HSP90 expression is blocked, whereas that of both HSP70 and IgG is still evident in cells after treatment, especially in bands at higher molecular masses. Western blotting for each type of protein is representative of at least three other experiments made on the same samples on different occasions.

The analysis made on cell lysates was also conducted on conditioned media (collected from the same cell cultures on which lysates were obtained, as reported above). Media were collected from duplicate wells, centrifuged for removing cell debris, extensively dialyzed, lyophilized and submitted to electrophoresis analysis followed by Western blotting with anti-HSP90, anti-HSP70 and anti-human IgG Abs (as above, for cell lysates). As shown in FIG. 3(B), no positivity for any of measured proteins was noted in media of control cells, whereas after stimulation with Grp94, mostly in complexes with IgG, there is a positive immune reaction for any proteins, in bands corresponding to those present in cell lysates. In particular, it was observed that the secreted species of HSP90, HSP70 and IgG were only those induced by treatments with Grp94, especially in complexes with IgG.

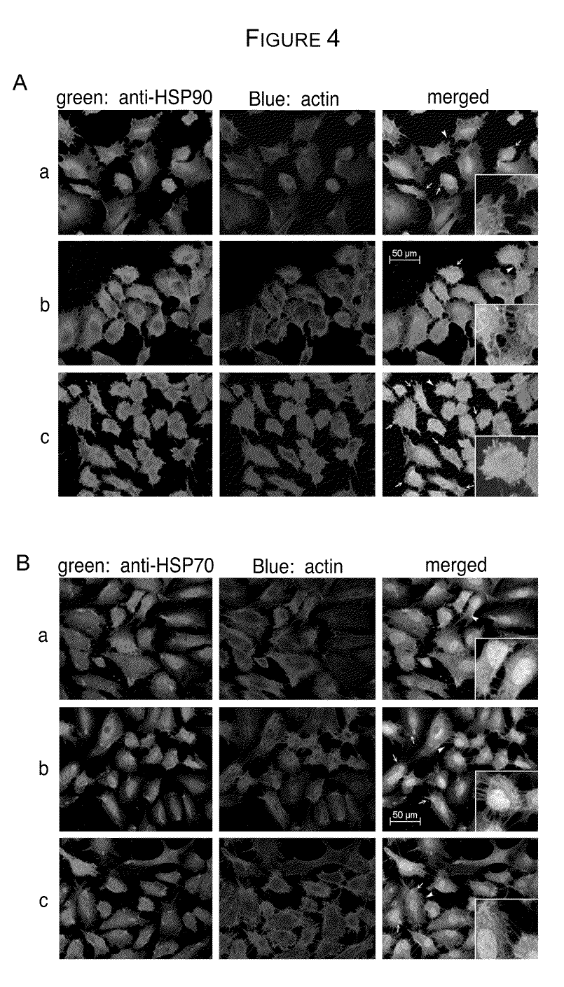

3. Morphological effects related to the enhanced expression of both HSP70 and HSP90 in HUVECs: hereafter are described in detail the method and results shown in FIG. 4.

Cells were cultured in serum-free medium both without any treatment (control, panels a) and with the addition of both Grp94 alone (10 ng/ml) (panels b) and Grp94 in complexes with IgG (panels c), as specified above. In FIG. 4(A) is shown the fluorescence (evidenced with confocal microscopy) due to anti-HSP90 Abs reacting with HSP90 in both control and treated HUVECs (panels a, b and c on left). Intensity of the fluorescence is increased in cells treated with Grp94, especially when present in complexes with IgG. In the latter case there is evidence of enhanced formation of podosomes (marked with arrows in panels of merged fluorescence) together with an extraordinary increase in actin filaments (central panels, blue fluorescence). Of interest is the co-location of HSP90 with actin, mostly evident in podosomes, underlined by the pale blue fluorescence due to merging of the green and blue fluorescence of HSP90 and actin, respectively (panels on right). In FIG. 4(B) is shown the fluorescence due to HSP70 following the addition to cell cultures of anti-HSP70 Abs, similarly to what reported for anti-HSP90 Abs. The fluorescence of HSP70 is also increased in treated (panels b and c) with respect to control HUVECs (panels a), although the intra-cellular dispersion of HSP70 is not so diffuse as that of HSP90. HSP70 is prevalently concentrated along the margins and at the leading edge of cells and does not show, unlike HSP90, a complete co-location with actin. Arrow heads in both (A) and (B) mark some of the most significant structural changes in the cell cytoskeleton (inserts in panels on right).

Although the effects of cell growth stimulation and differentiation towards the angiogenic phenotype of HUVECs could be interpreted as potential unwanted effects, since both predict the formation of new vessels that anticipate the development of more stable and severe vascular alterations, the same effects may nevertheless have very different consequences if extended to specific cell types other than vascular endothelial cells. In particular, since endothelial cells, mostly those of the umbilical cord, display the characteristics of cells of the innate immunity (they originate from a common ancestor in the bone marrow) (Wallin R. P. A. et al. Trends Immunol. 2002; 23:130-135), it is reasonable to think that effects observed in HUVECs could be extended on cells of immunity such as lymphocytes, in which proliferative and differentiation effects might be exploited to induce differentiation towards plasma cells, thus leading to the production of IgG with enhanced immune response.

To test this specific effect, experiments are carried out on human peripheral blood mononuclear cells (PBMCs) described hereafter.

4. Effects of stimulation of PBMC: PBMC were obtained from blood using the method of the Ficoll-Paque gradient. A sample of about 25 ml of buffy-coat (concentrated sample of red blood cells, white blood cells and platelets) was obtained after centrifugation of 250 ml of blood. The buffy-coat, containing about 300-600 million of PBMCs (1 million/ml of blood sample), was put in a 50-ml Falcon test-tube with the addition of a volume of RPMI (Roswell Park Memorial Institute) medium equal to half of the buffy-coat volume. The first step of PBMC separation included the preparation of the Ficoll solution in aliquots kept in the dark at +4° C. Five ml of buffy-coat were dropped on 4 ml of Ficoll solution put in Falcon test-tube (14-ml capacity). After centrifugation at 600×g for 30 min in swinging buket rotor, the white disk of PBMCs stratified between the upper solution of Ficoll and the lower one of plasma (red blood cells instead precipitate to the bottom), and was thus recovered with a Pasteur pipette. The fractions of PBMCs were mixed together in a Falcon test-tube (50-ml capacity) and fresh RPMI medium added up to the volume of 50 ml. After centrifugation at 600×g for 15 min, the supernatant was discarded and the pellet washed twice by adding the medium up to the volume of 50 ml, each washing being followed by centrifugation at 300×g for 10 min. After the last centrifugation, the pellet was re-suspended with RPMI medium up to 50 ml of volume.

The count of PBMCs was made on a diluted solution of PBMCs, mixing 50 μl of the starting solution in 250 μl (final volume) of Turk dye solution (in acetic acid). The cell count was made in a common cell-counting chamber.

In experiments aimed at measuring IgG concentrations in the medium, PBMCs are seeded at the concentration of 2×10⁶ cells/ml (1 ml/well) in a 24- or 48-well flat-bottomed plate (wells of 2 and 1 ml, respectively) incubated at 37° C., in a humidified 95% air, 5% CO₂ atmosphere for 2 weeks. Controls included triplicate wells containing medium only (or the diluent in which substances were dissolved, where appropriate), puromicine (100 μg/ml, final concentration, useful for detecting background Abs, i.e., Abs already present in circulation non specifically bound to blood cells), and a standard mitogen (pockweed mitogen, PWM) diluted 1:50 (v:v). Treatment included the addition of Grp94 (at final concentrations of 1, 10 and 100 ng/ml), both alone and in complexes with IgG, and IgG alone (at the final concentration necessary to give a 1:1 molar ratio with Grp94), after each solution was incubated at 37° C. for 1 h.

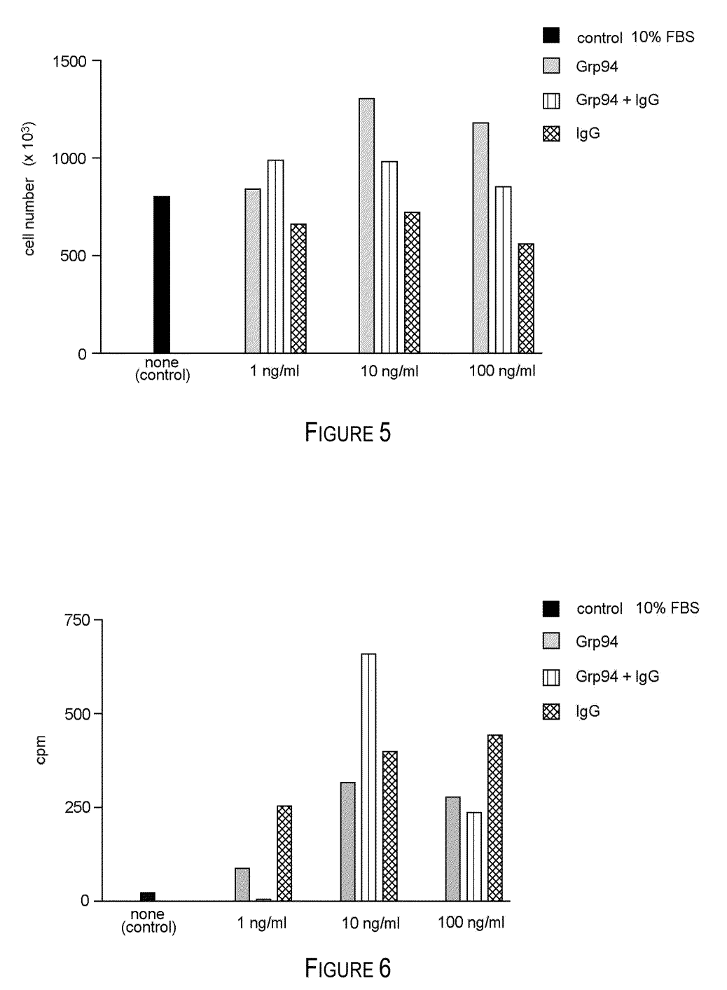

After 2-week incubation, cell cultures were examined at the optical microscope for assessing sterility, medium was collected and cells harvested. An aliquot of the cell suspension was used for counting cells (as specified above) whereas the remaining cell suspension was submitted to centrifugation (at 300×g for 10 min). The supernatant was stored at −20° C. until it was used for further analysis (measurements of IgG production), whereas the pellet was discarded. In FIG. 5 histograms represent the number of cells treated with both 10% FBS (taken as control) and with the indicated substances in absence of FBS. The stimulation induced by Grp94 alone was a dose-dependent effect up to the concentration of 10 ng/ml (increases of 5% and 63% with respect to the control value, for 1 and 10 ng/ml, respectively), whereas at 100 ng/ml of Grp94 the stimulation appeared to be slightly lower (47% increase with respect to the control value). A significant cell growth stimulation was noted at the lowest concentration of Grp94 with IgG (23% over the control value). Stimulation instead appeared to be reduced at higher concentrations of Grp94 with IgG (22.4% and 6%, respectively, at 10 and 100 ng/ml). This pattern closely resembles that displayed by growth factors that induce proliferation in a very narrow range of concentrations, above and below which a very little or no effect at all is observed. IgG alone did not induce any proliferative effect on PBMCs that were even reduced in number with respect to the control.

5. IgG production from PBMC: on a 96-well (200 μl each) flat-bottomed Optiplate (Packard) were laid down 50 μl of anti-human IgG Abs (5 μg/ml in NaHCO₃/Na₂CO₃ buffer, ph 9.6) that were allowed to dry out overnight. Afterwards, 200 μl of PBS with 3% BSA and 0.02% NaN₃ were added to saturate aspecific binding sites in Abs. Wells were then washed with PBS and the supernatant of each PBMC culture was added after serial dilutions in the PBS/BSA/NaN₃ solution. The calibration curve was made with a standard solution of human IgG (2 mg/ml) progressively diluted up to the concentration of 0.4 μg/ml. Control wells contained both the buffer solution without Abs and the buffer solution with Abs but without treatment solutions. After washing with PBS (200 μl), 50 μl of anti-human Ig-I¹²⁵ (1 μCi/ml dissolved in PBS/BSA/NaN₃ solution) was added to each well and allowed to rest for 2 h. The plate was then dried after additional washings and radioactivity measured after the addition of 30 μl of Microscint 20 in each well. Results of experiments of IgG measurements after 2-week incubation at 37° C. are reported in FIG. 6. The pattern of stimulation of IgG production by Grp94 is similar to that of Grp94 in complexes with IgG, in both cases the maximum of stimulation being reached at the concentration of 10 ng/ml. However, the entity of stimulation is much higher with Grp94 in complexes with IgG (659 cpm vs 316 cpm for Grp94 alone).

The foregoing description of embodiments of the present invention has been presented for the purpose of illustration and description. It is not intended to be exhaustive or to limit the invention to the particular forms disclosed. Obvious modifications and variations are possible in light of the above disclosure without departing from the spirit and scope of the present invention. The embodiments described were chosen to best illustrate the principles of the invention and practical applications thereof to enable one of ordinary skill in the art to utilize the invention in various embodiments and with various modifications as suited to the particular use contemplated. It is intended that the scope of the invention be defined by the claims appended hereto.

What is claimed is:

1-24. (canceled)

25. A Complex of Grp94 with human plasmatic immunoglobulins G (IgG), wherein said IgG are pre-immune and said complex has:
a stable binding between Grp94 and IgG at site(s) other than the antigen-binding site on IgG;
a resistance to denaturing conditions of boiling and reducing treatments;
a mass higher than 300 kDa measured on glycerol gradient (from 10% to 40%).

26. The complex of Grp94 with human plasmatic IgG according to claim 25, wherein Grp94 is either mammalian native protein or recombinant protein or fragment or mimetic thereof.

27. The complex of Grp94 with human plasmatic IgG according to claim 25, wherein IgG are an integer molecule.

28. The complex of Grp94 with human plasmatic IgG according to claim 25, wherein the mass of said complex is between 300 and 350 kDa.

29. The complex of Grp94 with human plasmatic IgG according to claim 25, wherein the molar ratio between Grp94 and IgG is from 0.5 to 1.0.

30. The complex of Grp94 with human plasmatic IgG according to claim 29, wherein the molar ratio between Grp94 and IgG is 1:1.

31. A complex of Grp94 with human plasmatic IgG according to claim 25 as immunomodulator for treating a subject in a need thereof.

32. The complex of Grp94 with human plasmatic IgG according to claim 31, for treating pathologies characterized by a deficit of the immune response.

33. The complex of Grp94 with human plasmatic IgG according to claim 32, wherein said pathologies are tumors, inflammatory diseases and pathologies characterized by the immune deficiency of different etiologies.

34. The complex of Grp94 with human plasmatic IgG according to claim 31, for treating pathologies characterized by an exaggerated immune response.

35. The complex of Grp94 with human plasmatic IgG according to claim 34, wherein said pathologies are autoimmune diseases.

36. The complex of Grp94 with human plasmatic immunoglobulins G (IgG), wherein said complex is obtainable by:
incubating Grp94 with human plasmatic pre-immune IgG at molar ratios comprised between 0.5 and 1.0; and
performing the incubation at temperatures between 30° C. and 40° C., for at least 1 h in an aqueous solvent, optionally buffered at pH between 6.5 and 7.4.

37. The complex of Grp94 with human plasmatic IgG according to claim 36, wherein said complex is obtainable by incubating Grp94 with human plasmatic pre-immune IgG at the molar ratio of 1:1 at the temperature of 37° C.

38. The complex of Grp94 with human plasmatic IgG according to claim 36, wherein said complex has:
a stable binding between Grp94 and IgG at site(s) other than the antigen-binding site on IgG;
a resistance to denaturing conditions of boiling and reducing treatments;
a mass higher than 300 kDa measured on glycerol gradient (from 10% to 40%).

39. The complex of Grp94 with human plasmatic IgG according to claim 36 as immuno-modulator for treating a subject in a need thereof.

40. A composition comprising a complex of Grp94 with human plasmatic IgG according to claim 25, in combination with pharmaceutically acceptable additives and carriers suitable for systemic and local administration, also in suitable controlled-release delivery systems.

41. A composition comprising a complex of Grp94 with human plasmatic IgG according to claim 36, in combination with pharmaceutically acceptable additives and carriers suitable for systemic and local administration, also in suitable controlled-release delivery systems.

42. A method for modulating the immune response in ex vivo experiments comprising the steps of:
obtaining a blood sample from a subject;
isolating cells of the immune system selected among peripheral blood mononuclear cells (PBMCs) from said blood sample; and
incubating said isolated cells, for at least 1 h, with Grp94-IgG complexes according to claim 25.

43. The method according to claim 42, wherein said isolated cells are a sub-population of cells selected on the basis of a specific antigen and/or membrane marker before the co-incubation with Grp94-IgG complexes.

44. The method according to claim 42, wherein said isolated cells are co-incubated at the concentration of 2 million/ml with Grp94-IgG complexes at concentrations of between 10 ng/ml and 10 μg/ml.

45. A method for detecting the presence of anti-Grp94 antibodies and/or anti Grp94-IgG complex antibodies comprising the step of:
obtaining a blood sample from a patient;
separating plasma or plasma-purified IgG fraction from said blood sample;
contacting said plasma or plasma-purified IgG fraction with Grp94-IgG complexes according to claim 25 and/or denaturated Grp94; and
detecting the anti-Grp94 antibodies and/or anti Grp94-IgG complex antibodies in the immunocomplexes obtained in the previous step.

46. The method according to claim 45, wherein said complexes of Grp94-IgG and/or denaturated Grp94 are attached to a suitable array substrate and said plasma or plasma-purified IgG fraction are labeled.

47. The method according to claim 45, wherein the amount of labeled IgG that binds the Grp94-IgG complexes and/or denaturated Grp94 are measured by detecting the label.

48. The method according to claim 45, wherein the presence of said antibodies is a marker of inflammatory conditions and/or tissue damage related to an altered immune response.

49. The method according to claim 45, wherein said antibodies are anti-Grp94 idiotypic antibodies.

50. The method according to claim 45, wherein said antibodies are directed against Grp94-IgG complexes, and are both idiotypic and anti-idiotypic in nature.

51. A diagnostic kit for the detection and quantitative measurement of plasma anti-Grp94 and anti Grp94-IgG complex antibodies for the method according to claim 45, comprising at least in one or more containers: a) complexes of Grp94-IgG, optionally labeled, immobilized on a suitable array substrate; b) denatured Grp94, optionally labeled, immobilized on a suitable array substrate; c) test anti-Grp94 antibodies optionally labeled; d) control plasma or plasma-purified IgG fraction; e) reagents for labeling plasma proteins or plasma-purified IgG fraction; f) reagents for detection of the immunocomplexes and a leaflet of instructions for use.
